(19) United States
(12) Patent Application Publication
(10) Pub. No.: US 2025/0037868 A1
(43) Pub. Date: Jan. 30, 2025

(54) Methods and Systems for Medical Prediction Using Collagen Fiber Architecture of Lesions

(71) Applicant: Picture Health, Inc., Cleveland, OH (US)

(72) Inventors: Haojia Li, Cleveland, OH (US); Nathaniel Braman, Cleveland, OH (US); Anant Madabhushi, Atlanta, GA (US)

(21) Appl. No.: 18/786,397

(22) Filed: Jul. 26, 2024

Related U.S. Application Data
(60) Provisional application No. 63/516,355, filed on Jul. 28, 2023.

(51) Int. Cl. G16H 50/20; G16H 20/10; G16H 30/00; G16H 50/70

(52) U.S. Cl. CPC G16H 50/20; G16H 20/10; G16H 30/00; G16H 50/70

(57) ABSTRACT

A machine learning system makes medical predictions based, at least in part, on pathomic features indicating collagen fiber organization extracted from pathology images that include lesions. The pathomic features are extracted from pathology images. The pathology images may be routine clinical images gathered in the course of diagnosis and treatment, such as hematoxylin and eosin (H&E)-stained whole slide images (WSIs) of tissue. The medical predictions may concern, e.g., the diagnosis, prognosis, genotype, or phenotype of a lesion, and may include disease-free survival (DFS) and overall survival (OS) of patients known or suspected of having malignant lesions. Methods for training a machine model to make, and for making, such predictions are also disclosed.

### CROSS-REFERENCE TO RELATED APPLICATIONS

This application claims priority to, and the benefit of, U.S. Provisional Patent Application No. 63/516,355, filed Jul. 28, 2023, the contents of which are incorporated by reference herein in their entirety.

### TECHNICAL FIELD

The invention relates to the fields of medical artificial intelligence and pathomics. More specifically, it relates to methods and systems for medical prediction using the collagen fiber architecture of lesions.

### BACKGROUND

The role of collagen in the genesis and metastasis of cancer is an area of active research. While the role of collagen differs depending on the type of cancer and other factors, cancer cells often promote collagen growth and modify the content and distribution of collagen. Collagen may form the major component of the tumor microenvironment and is known to have roles in cancer cell proliferation and invasion, tumorigenesis, and metastasis, to name a few. Collagen may also protect cancer cells from attack by the body's immune system.

When a patient has, or is suspected of having, a form of cancer, a blood or tissue sample is often taken for microscopic pathology examination, e.g., to make or confirm a diagnosis. In some cases, a solid tumor or suspected solid tumor may simply be excised and some of the tissue sent for microscopic pathology examination.

Historically, most pathology analysis of tissue samples has been qualitative in nature-a pathologist or another trained person examines the tissue sample and determines a diagnosis, prognosis, or other relevant information based on training and experience. Sometimes, qualitative analysis is supplemented by simple quantitative methods.

More recently, automated quantitative methods for analysis of pathology images have been developed. For example, in the field of pathomics, large amounts of quantitative data, called features, are extracted from images of tissue samples. Those features are analyzed by machine learning models to make medical predictions. Pathomic methods are sometimes described as a form of medical artificial intelligence.

U.S. Pat. No. 10,937,159, the contents of which are incorporated by reference herein in their entirety, describes a pathomic method and systems for generating a prognosis of disease-free survival for a patient demonstrating ER+ breast cancer by automated analysis of collagen fiber orientation using machine learning models.

### BRIEF SUMMARY

Aspects of the invention relate to methods and systems for using machine learning models to provide medical predictions based, at least in part, on pathomic features indicating collagen fiber organization extracted from pathology images that include lesions. The pathology images may be routine clinical images gathered in the course of diagnosis and treatment.

Systems and methods according to these aspects of the invention may, for example, provide a medical prediction for a particular patient using a machine learning model trained to make a medical prediction using pathomic features extracted from a whole slide image (WSI) including a lesion, such as a solid tumor. At least some of the pathomic features may relate to collagen fiber organization, such as collagen fiber orientation features. The medical prediction may be, e.g., for disease-free survival (DFS), overall survival (OS), or any other “endpoint” metric commonly considered in the treatment of cancers or other types of lesions. The medical prediction may also relate to the diagnosis, prognosis, genotype, or phenotype of the lesion.

Other aspects, embodiments, features, and advantages of the invention will be set forth in the following description.

### BRIEF DESCRIPTION OF THE DRAWING FIGURES

The invention will be described with respect to the following drawing figures, in which like numerals represent like features throughout the description, and in which:

FIG. 1 is a schematic flow diagram of a method for producing a medical prediction from a pathology image according to one embodiment of the invention; and

FIG. 2 is an illustration of a system that may be used to carry out a method like the method of FIG. 1.

### DETAILED DESCRIPTION

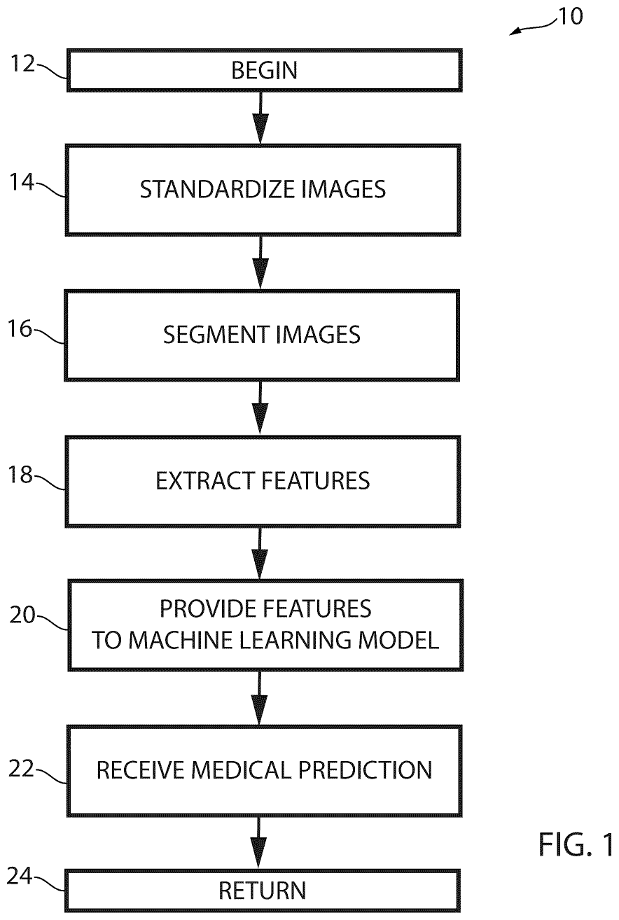

FIG. 1 is a flow diagram of a method, generally indicated at 10, according to one embodiment of the invention. Generally speaking, method 10 is a method for producing a medical prediction from a pathology image showing tissue in which a lesion is present. More specifically, as will be described below, method 10 extracts pathomic features indicative of collagen fiber organization in the pathology image and uses machine learning and machine learning models to generate medical predictions based, at least in part, on the extracted features.

As the term is used here, “pathology image” may refer to any type of image used in medical or research pathology that illustrates tissue. Most often, the pathology image will be a whole slide image (WSI) or portion thereof that has been stained appropriately for microscopic examination. One of the most common stains used in medical pathology is hematoxylin and eosin (H&E) stain, and the remainder of this description will assume that the pathology image is a WSI stained with H&E stain unless specifically indicated otherwise. While it is possible to use stains specific for collagen fibers, like Mason's Trichrome or AZAN Trichrome, as the description below will bear out, one advantage of method 10 and methods like it is that routine clinical pathology samples can be used. That is, the pathology images described here are usually taken from pathology samples that are acquired in the routine course of diagnosis or treatment, and no special effort need be taken in collection, preparation, staining, or any other typical task of processing a clinical pathology sample in order to use an image of that sample in method 10.

The tissue that is depicted in the pathology images would typically be tissue extracted from a patient demonstrating a lesion in a routine surgical procedure. That procedure may be, e.g., a biopsy or a full excision of the lesion. The surgical procedure may be undertaken for purposes of diagnosis (e.g., to confirm whether a lesion is benign or malignant), for resection or excision, or for any other clinical or research purpose. In keeping with routine clinical practice, the excised tissue would typically be prepared for microscopic pathology study by conventional processes, usually including one or more of fixation, dehydration, embedding, sectioning, and antigen retrieval, followed by staining. If images of pathology samples prepared for microscopy are not routinely taken at a particular hospital, clinic, laboratory, or other setting, image capture may be added to the routine processing steps. The pathology images described here may be taken at a standard magnification and a standard resolution. If the pathology images are not at a standard magnification or a standard resolution, they may be modified before further use in method 10 and other methods according to embodiments of the invention

In this description, the term “lesion” is used in the general sense to indicate any kind of injury to, or disease in, an organ or tissue that is discernible in a pathology image, e.g., a solid tumor extracted from a lung, a section of ovarian tissue demonstrating epithelial ovarian cancer, etc. While the most common type of lesion processed by method 10 and other methods and systems according to embodiments of the invention may be a solid (i.e., malignant) tumor of some kind, this description does not necessarily presume that a lesion is a solid tumor, because that may not be known with certainty when method 10 begins; i.e., the purpose of method 10, in some embodiments, may be to establish whether a lesion is benign or malignant.

Method 10 begins at 12 and continues with task 14. As method 10 begins, pathology images such as WSIs of tissue samples have typically been scanned by a slide scanner, photographed, or otherwise converted into digital pathology images. However, these images may need some element of preprocessing or standardization, e.g., to upsample or downsample them to a standard resolution, to crop, or to remove artifacts. In task 14, any necessary standardization or preparatory steps are undertaken. In some cases, a quality control process or application could be used, like that disclosed in U.S. Pat. No. 10,861,156, the contents of which are incorporated by reference in their entirety.

The pathology images may be in essentially any useable format, including Digital Communications in Medicine (DICOM) format, TIFF format, SVS format, JPG format, etc., and may be associated with metadata describing the nature of the study that produced the image, patient information, etc. In non-clinical uses of method 10, it may be necessary or desirable to anonymize data so as to protect patient identity.

For case in explanation, task 14 is shown as a part of method 10. However, in some embodiments, the function of task 14 may be performed routinely or automatically outside the scope of method 10. For example, pathology images entering a storage repository after capture by a scanner or other form of digitizer could be automatically standardized in format, cropped, etc. as part of the capture and storage process. Alternatively, all or a subset of the pathology images in a repository could be selected and standardized automatically or manually, either for general maintenance or in anticipation of a method like method 10. Method 10 then continues with task 16.

In some cases, the “preprocessing” steps performed on a pathology image may be a function of the file format in which it is stored. For example, in DICOM whole slide imaging, lower-resolution versions of a whole slide image are automatically computed and stored in a “pyramid” of image data that facilitates retrieval of image data at arbitrary resolutions.

In task 16 of method 10, the pathology image or images are segmented. “Segmentation” is a general term that, in this description, refers to any task or set of tasks that are performed in order to enable a machine to differentiate between the structures in a pathology image. For example, in order to extract and use features descriptive of the organization of collagen fibers in a WSI showing a lesion, a machine learning model may need to understand where the lesion is in the WSI, where the collagen fibers are, and where the other cellular structures, like the cellular nuclei, are. Segmentation may also focus on identifying a particular region or regions of the lesion, such as the lesion-associated stroma.

Segmentation may be performed in any number of ways, and different methods may be used in any one implementation of task 16. Some structures may be identified manually, while other structures may be identified automatically. Different structures may be identified in different ways, and/or using different machine learning models. Deep learning models, like convolutional neural networks (CNNs), may be used in segmentation. For example, a U-Net, a type of CNN particularly adapted for biomedical image segmentation, may be used to perform segmentation on an input pathology image.

As one example of the above, the borders of the lesion may be identified manually, the lesion-associated stroma may be segmented by a deep learning model, and the organization of collagen fibers may be measured by a computer-vision based approach. Manual input may be taken in any number of ways.

The segmentation of any particular type of structure in a pathology image may use one segmentation model or more than one type of segmentation model. For example, in identifying a particular structure, a low-resolution segmentation model could be trained to identify a general region of interest or one or more tissue types of interest within a larger image, and then a higher-resolution segmentation model could be used to identify particular structures, such as cells, within that region of the larger image. Of course, if a single segmentation model can provide adequate performance, then a single segmentation model can be used.

Additionally, different segmentation models may be used to identify different structures in the pathology images. For example, one segmentation model may be used to identify the neoplastic cells or nuclei of the lesion, a second model may be used to identify the stroma, and a third model may be used to segment the collagen fibers.

Prior to task 16, the machine segmentation model to be used in task 16 is specifically trained for the task, thus allowing the machine segmentation model to identify the structure or structures of interest. Typically, a cohort of pathology images with known segmentations would be used to train the segmentation model, and then the performance of the trained model would be evaluated using a second, different cohort of pathology images with known segmentations. Training typically continues until the performance of the machine segmentation model has improved to a sufficient threshold. An untrained model, by contrast, is typically unable to discern the structures of interest with any accuracy; thus, training represents a specific improvement in the performance of the machine for this particular task. Any training techniques known in the art may be used in embodiments of the present invention.

If different segmentation models are to be trained to identify different structures, the models may be trained separately or together. Different structures in, e.g., a WSI, may be visible at different resolutions, requiring different models to be trained using images at different resolutions. Augmentation techniques could be used, e.g., shifting the images vertically or horizontally to make the models more robust for varied data.

In some cases, techniques of “pre-training” or “transfer learning” may be used, i.e., using a pre-trained model to train another model. In general, any known techniques for increasing the robustness of machine learning models could be used in training the segmentation models.

Once the pathology image in question has been segmented, method 10 continues with task 18 and a number of pathomic features are extracted from the pathology image. Generally speaking, the term “feature” refers to any piece of quantitative information extracted from the pathology image or images, or a statistic of that quantitative information. In pathomics, features are typically extracted in great quantities, great enough that pathomic methods like method 10 cannot be performed merely in the mind, or by using manual computational tools. These features may in many cases be “sub-visual,” such that they cannot be observed by the human eye. The extraction may also involve computations too complex to compute by hand.

In executing task 18, the pathology image or images may be processed in piecewise-fashion by dividing the image, or at least the area of it to be processed, into tiles. For example, the use of 2000-pixel by 2000-pixel tiles at 40× magnification may be appropriate for areas both within and outside of the lesion. Additionally, within each tile, or within the whole of the pathology image if tiles are not used, the pathology image may be divided and portions grouped in a number of ways for feature extraction.

The most straightforward way of dividing and grouping may be to divide the pathology image or its tiles into neighborhoods and to extract features from each neighborhood. A neighborhood, as that term is used here, is a region or portion of the pathology image that is smaller than the whole, small enough that local feature values and trends can be observed. The size and shape of a neighborhood may vary from application to application and embodiment to embodiment. A neighborhood may be measured in image pixels or in real dimensions. For example, a particular neighborhood might be a 50 μm by 50 μm square in real dimensions. Neighborhoods may also be rectangular, triangular, pentagonal, hexagonal, etc. In some cases, neighborhoods may vary in size in the same image or, at least, in the same tile, with smaller neighborhoods defined in areas that are likely to include greater or more relevant change, or that otherwise require more granular examination with a smaller field of view.

As was noted briefly above, pathology images and tiles may be grouped and divided in other ways for feature extraction. Thus, a neighborhood might also be defined based on what it contains—e.g., a specific type of structure, cell, or tissue. For example, a neighborhood containing only a single type of tissue or a neighborhood at the intersection of two particular tissue types might be defined.

While any pathomic features may be extracted in method 10, typically at least a set of features indicative of collagen fiber organization is extracted. Collagen fiber orientation is one example of feature indicative of collagen fiber organization. Other examples of features indicative of collagen fiber organization include angles between adjacent collagen fibers, collagen fiber orientation relative to other structures and tissues of interest, and collagen density within a region or subregion. Examples of collagen fiber orientation relative to other structures and tissues of interest include fiber orientation relative to the boundary of a tumor nest, or fiber orientation relative to the orientation of the cells.

This description will use collagen fiber orientation as a particular example. As one example of a process of extracting features indicative of collagen fiber orientation, the orientation of collagen fibers is first detected. Following that, the degree of disorder of the fiber orientations can be calculated. To measure the disorder of fiber orientations, the orientations can be discretized into, e.g., 18 angle bins and a co-occurrence matrix constructed. An entropy feature indicative of the degree of disorder can then be extracted from the co-occurrence matrix.

This kind of entropy feature is usually considered in the context of neighborhoods of various scales. For example, the features used in method 10 may include the degree of collagen fiber orientation disorder in neighborhoods of 50 μm by 50 μm in the whole lesion, collagen fiber orientation disorder degree in neighborhoods of 100 μm by 100 μm at the lesion's leading edge, collagen fiber orientation disorder degree in neighborhoods of 150 μm by 150 μm in the normal tissue adjacent to the tumor tissue, etc.

The present inventors have surprisingly found that if collagen fiber orientation disorder is evaluated, the orientation disorder of fibers at the interface between the stroma and the nest of neoplastic cells (i.e., the tumor nest) may be more predictive of disease-free survival (DFS) than fiber orientation in other areas of the lesion. The present inventors have also found that evaluating collagen fiber orientation in a smaller field of view may be more predictive than if those same features are taken over a larger field of view. The 50 μm-by-50 μm neighborhoods may be optimal for making at least some types of predictions.

As was alluded to above, statistics of extracted features may also be used and may themselves be considered features. For example, it may be convenient to use the mean, maximum, minimum, variance, skewness, kurtosis, etc. of a particular feature in a particular neighborhood as input to a model. “Raw” features extracted from a pathology image may also be normalized or otherwise manipulated before further use.

In task 18, the same or similar features may also be extracted at different sizes or dimensional scales. For example, if neighborhoods are defined positionally, a first set of features or their statistics may be extracted from a single neighborhood in a pathology image, a second set of features or their statistics may be extracted from that neighborhood and all immediately adjacent neighborhoods, and a third set of features or their statistics may be extracted over the entire area of the lesion in the pathology image. The features taken at each scale may capture different information.

More generally, regions or structures in a pathology image may be grouped in particular ways for feature extraction, and pathomic features may be extracted with or without regard to their physical location in the pathology image. In other words, e.g., pathomic features relating to disorder of collagen fibers may be extracted from stromal tissue with or without regard to where those stromal tissues are located relative to tumor nests.

The precise ways in which structures or regions are grouped or divided for feature extraction will depend on the nature of the lesion and the nature of the medical prediction or predictions that are to be made. Those groupings or divisions may or may not be rooted in a particular type of structure or tissue. In some cases, the groupings or divisions used for feature extraction may be closely related to structures, tissues, or regions that are segmented in task 16 of method 10, although they need not be.

The above description focuses on a “neighborhood” as a contiguous area of a particular size. However, as was alluded to above, a “neighborhood” may be defined in other ways, such as a collection of structures of the same type. For example, one neighborhood may include all neoplastic cells, and a second neighborhood may include adjacent tissues. If neighborhoods are defined based on the type of cell or tissue, in addition to neoplastic cells vs. non-neoplastic cells, for example, epithelium, stroma, necrotic tissue, and tumor nests (i.e., densely-packed clusters of neoplastic cells) may all be considered to be individual neighborhoods. As may be apparent from the above, a neighborhood need not be contiguous across a pathology image.

In the description above, it was noted that features extracted from or close to the interface between neoplastic cells and surrounding cells and tissues may be outcome-predictive. In general, pathomic features may be collected from outside of the lesion, i.e., the so-called peri-lesional region. The term “peri-lesional” is positional or locational in nature and is not limited to a particular type of structures or tissues. The peri-lesional region may be defined in a number of ways, e.g., by morphological dilation of the lesional boundary in the pathology image or as a circle of fixed distance from a centroid of the lesion.

For purposes of this description, the stroma can be considered to be at least a portion of the peri-lesional region. In many cases, the collagen fibers of interest will be found in the stroma, and thus, the stroma will be the portion of the peri-lesional region of greatest interest.

In one embodiment, pathomic features, at least some of them relating to collagen fiber organization, may be extracted from two or three sets of neighborhoods or regions of a pathology image: in the peri-lesional region, at or near the interface between neoplastic lesional cells and surrounding cells, and squarely within the lesion. In another embodiment, pathomic features may be extracted at the lesional interface and in the peri-lesional region. In some embodiments, the peri-lesional region may be divided into zones or sets of neighborhoods, some closer to the lesional interface and others farther away from that interface, and sets of features may be extracted from each zone or set of neighborhoods.

Method 10 continues with task 20, and the features are provided to a trained machine learning model to output one or more medical predictions. For purposes of this description, a “medical prediction” is anything medically relevant that can be predicted by method 10 and other methods like it. Medical predictions may include, but are not limited to, the diagnosis of a disease or the classification of a disease; prognoses and predictions of disease progression; predictions of whether a particular lesion is likely to respond to a particular treatment; predictions of whether the apparent growth of a lesion during treatment represents a true progression of the underlying disease or a pseudo-progression caused by treatment; predictions of whether a particular patient is likely to experience a particular side effect, like hyper-progression, from a particular treatment; predictions of disease-free survival (DFS) or overall survival (OS) for a patient exhibiting a particular characteristic, like highly aligned collagen fiber organization in a lesion; and the like.

The trained machine learning model itself is typically a trained classifier, a machine algorithm that can associate specific feature values with specific predictions and/or outcomes. Any type of classifier may be used, depending on the nature of the medical prediction that is to be made, the nature of the features, and other factors. The classifier may be, e.g., a logistic regression or Cox proportional hazards model, a linear discriminant analysis (LDA) classifier, a quadratic discriminant analysis (QDA) classifier, a bagging classifier, a random forest classifier, a support vector machine (SVM) classifier, a Bayesian classifier, etc. A trained neural network may also serve as a classifier. In some cases, operators like a Least Absolute Shrinkage and Selection Operator (LASSO) may be used for feature regularization and selection.

The description above refers to a “trained” classifier as a machine learning model. In a typical scenario, a classifier for use in method 10 and other methods like it is first trained using a cohort of pathology images for which the patient outcome or information relative to the desired medical prediction is already known. For example, if the desired medical prediction is whether or not a particular lesion is benign or malignant, the classifier would be trained on a cohort of pathology images with pathologically confirmed status of malignancy. If the desired medical prediction is whether or not a particular lesion will respond to a particular treatment, the classifier would be trained with a cohort of pathology images from patients who received that treatment and with available response outcome (a mix of responders and non-responders). If the desired medical prediction is whether a patient with a lesion having particular level of collagen fiber orientation disorder (CFOD) is likely to experience a short period of DFS or a long period of DFS, the classifier would be trained with a cohort of pathology images in which the outcome, such as DFS, is known in each case. As was noted above, other outcome measures, like overall survival (OS) may be used.

As a general matter, the training cohort would include both positive and negative samples, i.e., the outcomes represented in the cohort would be balanced. During training, the general flow of method 10 would be observed: the training images would be segmented, pathomic features would be extracted, and one or more features would be associated with the known outcome by the classifier. Once trained, the classifier would be validated using a new cohort of images, not previously presented to the classifier, in which the outcome relative to the desired medical prediction is also known. The validation cohort would generally also include positive and negative samples.

The receiver operating characteristic curve (ROC) and the area under the curve (AUC) are typically used to determine the ability of the trained machine learning model to make correct medical predictions. If the AUC is too low, remedial steps may be taken. Those remedial steps may include training a new machine learning model with more training data, using different combinations of pathomic features, etc. In some cases, the AUC for a machine learning model may be compared with the AUC for a trained person in making the same sort of medical prediction. For example, when possible, the machine learning model's predictive performance on the validation cohort of pathology images may be compared to the performance of a trained person examining the same set of pathology images. Of course, ROC and AUC relate to the performance of a machine learning model itself. In other cases, metrics that are more patient-centered may be used, such as concordance index, hazard ratio, and other metrics used in survival analysis.

Ultimately, the features provided to the trained machine learning model in task 20 of method 10 may be any that are found to be predictive for the intended medical prediction. The most stable and robust set of pathomic features for use in method 10 for a particular medical prediction may be chosen prior to the execution of method 10 in some cases. For example, a cohort of pathology images for which the outcome relative to the desired medical prediction is already known could be used to explore and to validate which set of pathomic features is most stable and robust for that particular medical prediction in the context of method 10. If desired, and if enough data is available, this approach could be extended to determining which type(s) of classifiers to use as the machine learning model(s) in task 20 of method 10.

Some types of classifier, like the LASSO regularized classifier, inherently include methods for reducing the feature set to the least redundant and most predictive feature set. If the classifier itself does not include that capability, other techniques may be used, particularly during the training of the classifier, to identify a subset of “top” features to be leveraged by the model according to their relevance, redundancy, and/or stability relative to the medical prediction at hand, such as the Wilcoxon rank-sum test, the maximum relevance, minimum redundancy (mRMR) approach, random forests, or forward selection, to name a few possible techniques.

One of the strengths of pathomic and machine-learning-based approaches is that machine learning models may make connections that human observers, however well trained, would not. For that reason, the nature of the features that are provided to the machine learning model in task 20 is not particularly limited. For example, if the objective is to predict DFS based on CFOD, pathomic features other than those related to CFOD may be used. For example, features relating to the nuclear organization of lesional cells may be used, as may graphs of those cells. See, e.g., U.S. Pat. No. 11,055,844, the contents of which are incorporated by reference herein in their entirety.

Features other than pathomic features may also be considered in some embodiments of method 10 and other methods according to embodiments of the invention. For example, if other types of medical images of the same lesion are available (e.g., computed tomography (CT) images, magnetic resonance imaging (MRI) images, X-ray images, positron emission tomography (PET) scan images, etc.) radiomic features could be extracted from those medical images and a combination of pathomic and radiomic features provided to a trained machine model.

Although much of the above description refers to a single machine learning model being used to make a prediction, task 20 may involve the use of any number of models. It may be advantageous to use separate, individually trained models if, for example, features are taken at different scales of size, or if different types of features are used to make a single prediction (e.g., several different types of pathomic features, or a combination of pathomic and radiomic features, etc.). Any group or type of features may be provided to any model trained to accept those features. Of course, if a single model that accepts multiple types of features provides adequate performance for the particular application, then a single model may be used. Different models may be of different types, i.e., they may use different algorithms to provide their predictions.

In this description, unless the term “model” is qualified in such a way as to indicate its nature (e.g., a machine learning model, deep learning model), the term should be interpreted more broadly. For example, a nomogram is a type of model that may be used in and with embodiments of the invention. Nomograms may be used with the medical predictions generated by method 10 and output as a part of descriptive clinical reports or in other ways. A nomogram might, for example, be used to compare or combine the predictions from two or more machine learning models. A simple, non-trainable combination strategy leveraging features of import should also be considered a model. For instance, the averaging or summing of several key features to derive an aggregate score would be an example of a simple model.

As those of skill in the art will understand, machine learning models, which include deep learning models, produce an output that typically cannot be replicated in the mind or using simple computational tools like pen and paper. In fact, with machine learning models, it may not be immediately possible to understand why the machine is classifying a particular pathology image in a particular way. Deep learning models, in particular, are infamous for their inscrutability. In many cases, it may only be possible to choose which loss function or functions the machine learning model attempts to optimize during training and use and to judge the correctness of the machine learning model's output during training, when data with known outcomes is used.

The prediction provided in task 22 of method 10 may be relatively basic: a probability or risk score and, in some cases, a confidence score or interval for the prediction. That form of output may be suitable for many applications, including research applications, like in pharmaceutical research and clinical trials. A prediction might also be converted to a simplified form before it is provided to the user. For instance, a continuous risk score obtained from a predictive model may be reported to a user as either a categorical “high risk” or “low risk” indication determined by whether the risk score exceeds some predetermined cutoff threshold value.

In addition to the prediction itself, it is often helpful to provide information, in task 22 or otherwise, that can be used to understand the prediction, its meaning, and its use. When machine learning tools provide obscure predictions for even more obscure reasons, clinicians may have reservations about relying on those predictions. In fact, relying on medical predictions with obscure bases may entail considerable clinical risk. For that reason, the prediction may be presented in a clinical or other type of report that includes the prediction with enough context to understand it. The report may be a standard-form report, either in print or electronic form, that can be reviewed by, e.g., a pathologist, oncologist, pulmonologist, etc. That report could contain, for example, a sample of the pathology images used to create the segmentation; a visualization or visualizations of the segmentation or masks/intermediates used to create the segmentation; feature values or elements descriptive of the feature values, like feature statistics or plots illustrating the features; the medical prediction; a confidence measure or interval for the prediction; and suggestions for follow-up study or action. The information on the report may be drawn from multiple sources, including a patient's medical chart. In some cases, the report may include the medical prediction along with the trained person's qualitative assessment of the pathology images on which the prediction was based. If a system implementing method 10 is in clinical use, the records, including the medical prediction and any reports associated with it, may be stored in an Electronic Health Record/Electronic Medical Record (EHR/EMR) system.

A “raw” probability or risk score output as a medical prediction in task 22 of method 10 may also be supplemented by visualizations, such as “heat maps” or other such indicators of a feature or features. For example, a “heat map” indicating the degree of CFOD could be superimposed on a tile or tiles from the original pathology image, giving the reader a visual indication of the degree of disorder over a particular area to support the medical prediction that is provided in task 22. Other types of visualizations may also be provided. For example, if the medical prediction that is output in task 22 is a simple classification of the patient (or the lesion) as “high risk” or “low risk,” a visualization like a Kaplan-Meier curve may be provided to illustrate the impact of that classification on overall survival.

In an EHR/EMR system, the prediction generated by the trained model may be used in various ways, including to generate pop-up alerts triggered in response to certain events. For example, alerts may inform a clinician that the pathomic study indicates the patient to have higher risk for a particular condition, a greater likelihood of response or non-response to a particular treatment, a greater risk of side effects with a particular treatment, etc.

In addition to the reports, intermediate products of method 10, like information on any manual input as to a region of interest and the encoding of that input, the segmentation, feature data, etc., may also be stored in the EHR/EMR if desired, either for further use and study, or simply to provide a record of the basis for the medical prediction that is provided in task 22.

Method 10 returns at task 24.

The tasks of method 10, or any subset of them, may be encoded in a set of machine-readable instructions that are interoperable with a machine to cause that machine, or a collection of interconnected machines, to perform the tasks of method 10. In essence, method 10 may be encoded as software to be run on a machine, typically a computer.

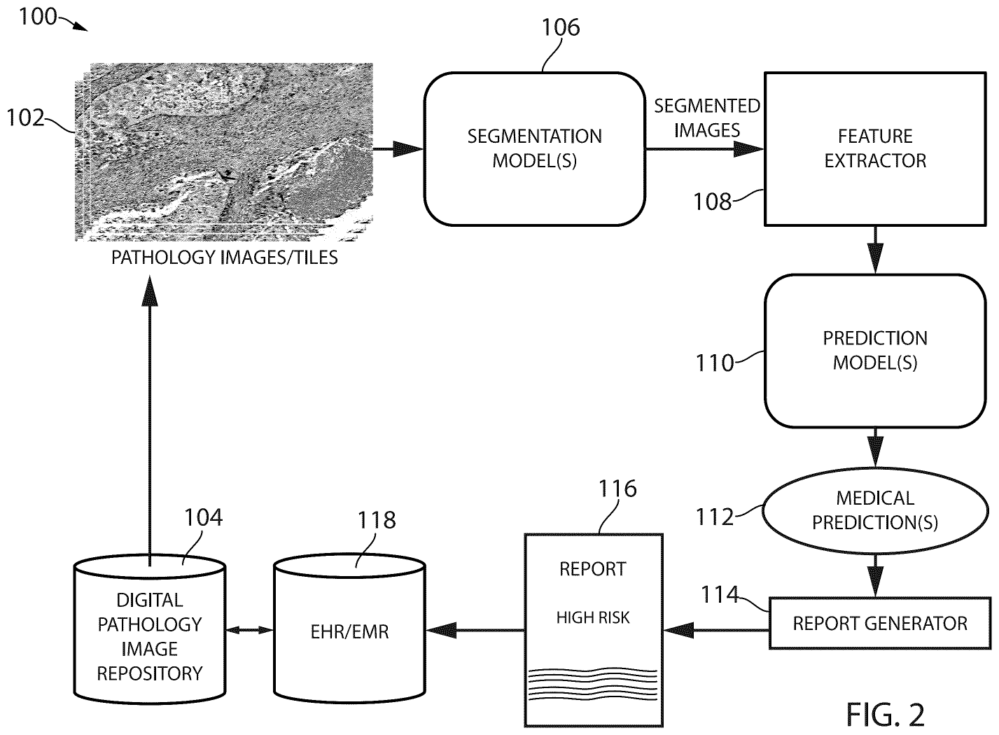

FIG. 2 is an illustration of a system, generally indicated at 100, adapted to carry out methods like method 10. In system 100, a set of pathology images or image tiles 102 is accessed from a digital pathology image repository 104 and provided to a segmentation model or models 106. While the pathology image, images, or image tiles 102 are typically in a viewable image format, there is no absolute requirement that that be so. Instead, a dataset extracted from the pathology images 102 that is specially configured or adapted for the use of a particular segmentation model 106 may be provided and used by the segmentation model 106.

Typically, system 100 of FIG. 2 is implemented on at least one computing system having at least one processor, although system 100 may be implemented on any number of interconnected or networked computers or processors. The segmentation model or models 106 may be present and executed on the same physical machine as the digital pathology image repository 104 or on a different machine entirely.

Due to the nature of segmentation operations, the segmentation models 106 and segmentation tasks 16 may, in some embodiments, be performed on a different processor, or a different type of logic device, than other operations of method 10 and system 100. For example, the segmentation model 106 may be implemented using one or more graphics processing units (GPUs), one or more field-programmable gate arrays (FPGAs), one or more application-specific integrated circuits (ASICs), instead of being implemented on a standard central processing unit or microprocessor. Segmentation models 106 may also be implemented in the cloud, i.e., via a network-accessible collection of computing systems designed for individual or shared use, that are presumably remote from the operator of system 100 and/or other components of it. The advantage of cloud computing systems is that, typically, much more computing power is available than if system 100 were run on one or several standalone machines, and that computing power can be recruited as needed to implement system 100 efficiently in a way that provides fast results. Although one segmentation model 106 is shown in the view of FIG. 2, as was described above, any number of segmentation models 106 could be used for any number of reasons.

Once segmentation is complete, a feature extractor 108 extracts pathomic features from the pathology image, images, or image tiles 102. The feature extractor 108 may be implemented on the same physical machine as the other components or on a different machine interconnected or networked to the other machines. The feature extractor 108 may use standard microprocessors/CPUs, or it may use GPUs, FPGAs, or ASICs, to name but a few options.

The extracted features are provided to a prediction model or models 110 that output one or more medical predictions 112. The prediction model or models 112 may be implemented on the same physical machine as the other components or on a different machine interconnected or networked to the other machines. The prediction model or models 110 may use may use standard microprocessors/CPUs, or they may use GPUs, FPGAs, or ASICs, to name but a few options. As was noted above, if the physical machines are cloud-based, as many CPU cores, GPUs, etc. as needed may be recruited to implement the prediction models 110 efficiently.

In some embodiments, the prediction model(s) 110 may be the last component of system 100; nothing more may be required. However, FIG. 2 illustrates a report generator 114, which is a module or algorithm that generates a clinical or research report 116 for the use of researchers or clinicians. As was described briefly above, that report 116 may include the “raw” medical predictions 112, classifications based on those predictions, intermediate products of method 10 and system 100, and contextual or interpretive information that may be useful in understanding the predictions, like heat maps, Kaplan-Meier curves, etc. In some cases, depending on the nature of the predictions and the desires of the operator of system 100, the report generator 114 may be a treatment plan generator that offers, as a part of the report 116 or separately, a personalized treatment plan for that patient. A personalized treatment plan may advise that a particular patient be administered a particular drug or drug regimen for a particular duration, or be subjected to one particular medical treatment instead of another. For example, in early-stage cancers, a personalized treatment plan may recommend surgical excision of a solid tumor, followed by a particular chemotherapy or chemotherapies as adjuvant treatment or, alternatively, neoadjuvant chemotherapy treatment followed by excision. A treatment plan may also indicate whether a particular drug or therapy is likely to generate a response in a particular patient, based on the medical prediction or predictions. For example, a treatment plan may indicate whether an immune checkpoint inhibitor (ICI), such as nivolumab, is likely to generate a response in a particular patient.

The report 116 may be stored in and used by an EHR/EMR system 118, which may or may not be in communication with the digital pathology image repository 104.

Depending on the complexity of the feature set, the complexity of the medical prediction that is to be made, and other factors, the medical prediction 112 and any reports 114 derived from it may be presented to researchers or clinicians in real time or at some later point. A real-time implementation may, for example, provide a response within 1-15 seconds of when the user first indicates that a medical prediction 112 is desired. Outputs of method 10 and system 100 may also be treated essentially as laboratory tests are in clinical medicine, to be reported back according to a priority assigned by a clinician (e.g., “stat” lab results in less than 60 minutes in more than 90% of situations). If a higher priority is assigned to a particular patient or set of pathology images 102, more computing resources could be recruited in executing method 10 for that set of pathology images 102.

While both method 10 and system 100 have been described here as having sequential steps, or having elements that are used sequentially, that need not be the case in all embodiments. As those of skill in the art will appreciate, system 100 may serve many patients and analyze many pathology images 102 at the same time, instantiating and running many instances of method 10 in parallel. Each individual instance of method 10 may have the same objective and/or provide the same type of medical prediction, or different instances may provide different medical predictions based on different extracted features or different models 106, 110.

It is also possible for method 10 to be executed discontinuously. For example, in some cases, any time a pathology image 102 is entered into a repository 104 with metadata indicating a lesion suspected of being a particular type or having particular characteristics, that pathology image 102 may be segmented and the segmentation stored until a medical provider calls up the pathology image 102 and requests a medical prediction. Similarly, a whole-slide imaging device or an image quality-control software package may have a segmentation module or circuit that automatically segments pathology images 102 as they are digitized or initially processed and stores that segmentation. In that case, the task of the segmentation model(s) 106 may be to produce a refined segmentation suitable for the medical prediction at hand, rather than to perform the kind of gross segmentation that has already been completed by another device or module.

Finally, while an implementation of system 100 on “generic” hardware is described above, in some cases, system 100 could be implemented on a single purpose-built machine or set of machines. The machine would have, e.g., enough CPUs, GPUS, memory, and storage space to handle the volume of medical prediction expected in a particular installation. Such a purpose-built implementation may be useful, e.g., when privacy concerns or other issues dictate an on-site installation of system 100.

The methods and systems described here are applicable to a broad range of lesions and cancers, including colorectal cancer, esophageal cancer, gliomas, head and neck cancers, lung cancer, ovarian cancer, pancreatic cancer, renal cancer, breast cancer, and cervical cancer.

While the invention has been described with respect to certain embodiments, the description is intended to be exemplary, rather than limiting. Modifications and changes may be made within the scope of the invention, which is defined by the appended claims.

What is claimed is:

1. A method, comprising:
extracting features from a pathology image of or including a lesion, at least some of the features being related to collagen fiber organization in the pathology image;
providing the extracted features to a machine learning model trained to make medical predictions based at least in part on the extracted features; and
receiving a medical prediction based on the extracted features from the machine learning model; and
outputting the medical prediction.

2. The method of claim 1, wherein the medical prediction concerns disease-free survival (DFS) or overall survival (OS).

3. The method of claim 1, wherein the medical prediction concerns whether or not the lesion will respond to a particular treatment.

4. The method of claim 1, wherein the medical prediction concerns a diagnosis, a genotype, or a phenotype of the lesion.

5. The method of claim 1, wherein the lesion is segmented in the pathology image.

6. The method of claim 5, wherein said extracting further comprises extracting the features near an interface of the lesion with surrounding tissue.

7. The method of claim 5, wherein said extracting further comprises extracting the features from an area within the lesion.

8. The method of claim 5, wherein said extracting further comprises extracting at least some of the features from a peri-lesional area around the lesion.

9. The method of claim 1, wherein said extracting further comprises extracting at least some of the features in a stroma from or surrounding one or more different regions or types of structures or tissues in the pathology image.

10. The method of claim 9, wherein said extracting further comprises extracting at least some of the features from a first portion of or location within the stroma.

11. The method of claim 10, wherein said extracting further comprises extracting at least some of the features from a second portion of or location within the stroma.

12. The method of claim 9, wherein the regions or types of structures or tissues segmented in the pathology image comprise: one or more of neoplastic cells, non-neoplastic cells, epithelium, stroma, necrotic tissue, or tumor nests.

13. The method of claim 1, wherein the features comprise an average degree of collagen fiber organization disorder in neighborhoods of a first size measured across the lesion, a standard deviation of collagen fiber organization disorder in neighborhoods of a second size at a leading edge of the lesion, or minima of collagen fiber organization disorder degree in neighborhoods of a third size measured across the lesion.

14. The method of claim 1, wherein the features comprise a statistic of collagen fiber organization disorder in at least one neighborhood of the pathology image.

15. The method of claim 1, wherein the pathology image comprises a hematoxylin and eosin (H&E)-stained whole slide image of a pathology sample.

16. A machine-readable medium encoded with instructions that, when executed by the machine, cause the machine to perform the method of claim 1.

17. An apparatus, comprising:
a memory and a processor coupled to the memory, the processor and the memory implementing a machine model trained to provide a medical prediction based on features extracted from pathology images including a lesion, at least some of the features being related to collagen fiber organization in the pathology image.

18. The apparatus of claim 17, wherein the medical prediction concerns disease-free survival (DFS) or overall survival (OS).

19. The apparatus of claim 17, wherein the medical prediction concerns whether or not the lesion will respond to a particular treatment.

20. The apparatus of claim 17, wherein the medical prediction concerns a diagnosis, a genotype, or a phenotype of the lesion.

21. The apparatus of claim 12, wherein the machine model comprises a classifier trained to provide the medical prediction based on the extracted features.

22. The apparatus of claim 21, wherein the trained classifier comprises a deep learning classifier.
